(19) United States
(12) Patent Application Publication
(10) Pub. No.: US 2018/0291098 A1
(43) Pub. Date: Oct. 11, 2018

(54) ANTI-NRG1 (HEREGULIN) ANTIBODIES AND USES THEREOF

(71) Applicants: INSERM (Institut National de la Sante et de la Recherche Medicale), Paris (FR); Universite de Montpellier, Montpellier (FR); Institut Regional du Cancer de Montpellier, Montpellier (FR)

(72) Inventors: Charline Ogier, Montpellier (FR); Christel Larbouret, Montpellier (FR); Andre Pelegrin, Montpellier Cedex 5 (FR); Thierry Chardes, Montpellier Cedex 5 (FR)

(21) Appl. No.: 15/767,003

(22) PCT Filed: Oct. 26, 2016
(86) PCT No.: PCT/EP2016/075740

§ 371 Date: Apr. 9, 2018

(30) Foreign Application Priority Data
Oct. 27, 2015 (EP) 15306715.2

(51) Int. Cl. C07K 16/28; C07K 16/32; A61P 35/00

(52) U.S. Cl. CPC C07K 16/28; C07K 16/32; A61P 35/00; A61K 39/00; C07K 2317/732; C07K 2317/76; C07K 2317/92; C07K 2317/30

(57) ABSTRACT

The disclosure relates to human anti-NRG1 neutralizing monoclonal antibodies that do not interfere with the NRG1 binding to the HER3 receptor and uses thereof. More particularly, an isolated human monoclonal antibody comprising a heavy and light chain variable regions with specific CDRs defined by their sequences is disclosed.

### FIELD OF THE INVENTION

The invention provides new anti-neuregulin 1 (NRG1) antibodies and uses thereof.

### BACKGROUND OF THE INVENTION

Neuregulin 1 (NRG1) signalling, which can occur through either the HER3 (ErbB3) or HER4 (ErbB4) receptor, can trigger multiple signaling cascades including the PI3K/Akt, PKC, MAPK and the Ras signaling pathways. Furthermore, inhibition of NRG1 signaling results in the delay or prevention of tumor relapse or recurrence after treatment with a therapeutic agent.

Anti-NRG1 antibodies that inhibit NRG1 induced signaling are useful in the treatment of cancers associated with NRG1 signaling, including autocrine NRG1 signaling. For instance, NRG1 autocrine signaling has been shown to regulate lung epithelial cell proliferation and has been implicated in insensitivity of NSCLC to EGFR inhibitors (Hegde et al., 2013).

International patent application WO 2013/025853 thus provides neuregulin antibodies and methods for using the antibodies in treating diseases or disorders, such as cancer. Said patent application describes neutralizing anti-NRG1 antibodies which binds to the EGF domains of NRG1α and NRG1β and inhibits the binding of NRG1 to HER3 and/or HER4.

Moreover, pancreatic Ductal Adenocarcinoma (PDAC) has a dramatic outcome, only palliative therapy is available and the 5-year survival rate is lower than 5%. A recent study has discovered the relationship between the receptor HER3, microenvironment cells like cancer-associated fibroblasts (CAF) and ligands expression such as NRG1 (Liles et al., 2011).

Therefore, there exists an unsatisfied need for simultaneously targeting NRG1 expressed by stroma cells (such as CAF) and inactivating HER3 and HER4 receptors following the binding of NRG1 to these receptors present at the surface of cancer cells (such as pancreatic tumour cells) in order to both inhibit tumor growth and tumor invasion and thus prevents tumor relapse or recurrence.

### SUMMARY OF THE INVENTION

In a first aspect, the invention relates to an isolated neutralizing monoclonal antibody that specifically binds to the human neuregulin 1 (NRG1) without interfering with NRG1 binding to HER3 receptor.

In a second aspect, the invention relates to an isolated anti-NRG1 monoclonal antibody or a fragment thereof comprising a heavy chain variable region comprising SEQ ID NO: 3 in the H-CDR1 region, SEQ ID NO: 4 in the H-CDR2 region and SEQ ID NO: 5 in the H-CDR3 region; and a light chain variable region comprising SEQ ID NO: 6 in the L-CDR1 region, SEQ ID NO: 7 in the L-CDR2 region and SEQ ID NO: 8 in the L-CDR3 region.

In a third aspect, the invention relates to an antibody or a fragment thereof according to the invention for use as a drug.

In a fourth aspect, the invention relates to an antibody or a fragment thereof for use in a method for treating cancer, especially pancreatic cancer.

### DETAILED DESCRIPTION OF THE INVENTION

The invention is based on the generation and characterization of one neutralizing anti-NRG1 antibody which binds to the extracellular domain (ECD) of NRG1 but not to the EGF domains of NRG1α and NRG1β. This neutralizing anti-NRG1 antibody inhibits NRG1-induced activation of HER3 receptor signaling but without interfering or blocking with NRG1 binding to HER3. Additionally, the inventors shown that said antibody not only binds to circulating NRG1 but also to NRG1 already bound to HER3. These both properties allow to take benefit from antibody effector functions such as antibody dependent cellular cytotoxicity (ADCC) on the contrary to the previously disclosed neutralizing anti-NRG1 antibodies.

The monoclonal antibody specific of NRG1 (also referred as anti-NRG1 mAb) selected by the inventors is able to inhibit pancreatic tumor cell proliferation and migration by inhibiting HER3 signaling pathways. In vivo study demonstrated the efficacy of said anti-NRG1 mAb on tumor growth. Interestingly, this mAb is further able to decrease pancreatic cell proliferation incubated with NRG1 expressing CAF conditioned medium.

### Definitions

Throughout the specification, several terms are employed and are defined in the following paragraphs.

The terms “Neuregulin” (NRG) or “Heregulin” are used interchangeably and have their general meaning in the art. The neuregulin cytokine family is comprised of four genes that encode a large number of secreted or membrane-bound isoforms. Thus, there are four known members of the neuregulin family, NRG1, NRG2, NRG3, and NRG4. The NRG1 transcript undergoes extensive alternative splicing resulting in at least 15 different iso forms. All active iso forms share an Epidermal Growth Factor (EGF)-like domain that is necessary and sufficient for activity by interacting with the ErbB family of tyrosine kinase receptors including Her1 (EGFR, ErbB1), Her2 (Neu, ErbB2), Her3 (ErbB3), and Her4 (ErbB4).

The terms “Neuregulin 1” (NRG1) is one of four proteins in the neuregulin family that act on the EGFR family of receptors. Neuregulin 1 is produced in numerous isoforms by alternative splicing, which allows it to perform a wide variety of functions. All NRG1 iso forms, divided in 3 distinct types of NRG1, contain an EGF-like domain that is required for their direct binding to the HER3 and/or HER4 receptor tyrosine kinases.

Within the context of the invention, the term “NRG1” refers to Type 1 NRG1 (also known as Heregulin) including alpha-splice variant (NRG1α1) and beta-splice variant (NRG1β1) that differ in the C-terminal portion of the EGF-like domain. The term “Neuregulin 1 alpha 1” (NRG1α1) refers to a particular splice variant of Type 1 NRG1. The naturally occurring human NRG1α protein has an aminoacid sequence of 640 amino acids provided in the UniProt database under accession number Q7RTV8.

The term “Neuregulin 1 beta 1” (NRG1β1) refers to a particular splice variant of Type 1 NRG1. The naturally occurring human NRG1β protein has an aminoacid sequence of 645 amino acids provided in the GenBank database under accession number NP_039250.2.

The term “anti-NRG1 antibody” refers to an antibody directed against NRG1 and that is capable of binding NRG1 with sufficient affinity such that the antibody is useful as a diagnostic and/or therapeutic agent in targeting NRG1.

The term “specifically binds to” means that an antibody only binds to the antigen of interest, e.g. NRG1, as assessed using either recombinant forms of the proteins, epitopes therein, or native proteins present on the surface of isolated target cells and does not exhibit cross-reactivity to other antigens. Accordingly, “an antibody that specifically binds to NRG1” does not bind to the other members EGF-family of proteins such as EGF, HB-EGF or AREG as well as to other members of the neuregulin family, Type II or Type III NRG1 (also known as SMDF), NRG2, NRG3, and NRG4.

According to the invention, the terms “antibody” or “immunoglobulin” have the same meaning, and will be used equally in the invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

According to the invention, the term “chimeric antibody” refers to an antibody which comprises a VH domain and a VL domain of a non-human antibody, and a CH domain and a CL domain of a human antibody. According to the invention, the term “humanized antibody” refers to an antibody having variable region framework and constant regions from a human antibody but retains the CDRs of a previous non-human antibody.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond. The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin. The term “Fab” refers to an antibody fragment having a molecular weight of about 50,000 and antigen binding activity, which is obtained by cutting a disulfide bond of the hinge region of the F(ab′)2. A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2. The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils.

As used herein, the terms “neutralizing antibody” refers to an antibody that blocks or reduces at least one activity of a polypeptide comprising the epitope to which the antibody specifically binds. A neutralizing antibody reduces an activity in vitro and/or in vivo. Typically, the neutralizing anti-NRG1 antibody according to the invention inhibits NRG1-induced activation of HER3 and/or HER4 receptor signaling. As demonstrated below, said neutralizing anti-NRG1 antibody inhibits NRG1-induced HER3 phosphorylation and then the HER3 downstream signaling pathways (which can be assessed by a phosphorylation assay for instance assessing the phosphorylation of HER3 and/or the phosphorylation of AKT and MAK assays as described in the section Examples). As also demonstrated, said neutralizing anti-NRG1 antibody inhibits NRG1-induced HER4 phosphorylation. The neutralizing anti-NRG1 antibody according to the invention also inhibits in vitro and in vivo the proliferation of cancer cells such as BxPC-3 (a pancreatic cancer cell line) and MCF7 cells (a breast cancer cell line) (which can be assessed by a wound healing assay, a cell viability assay and/or a proliferation assay or by xenografts experiments as described in the section Examples) but also targets the tumoral microenvironment by blocking the tumorigenic role of stromal cells.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, melanoma, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

Antibodies of the Invention:

In a first aspect, the invention thus relates to an isolated neutralizing monoclonal antibody that specifically binds to the human neuregulin 1 (NRG1).

In one embodiment, said neutralizing antibody specifically binds to the human NRG1 without interfering with NRG1 binding to HER3 receptor.

In a particular embodiment, said neutralizing antibody specifically binds to the extracellular domain (ECD) of NRG1.

In one embodiment, said antibody specifically binds to the peptide of SEQ ID NO: 1 derived from NRG1 (peptide ranging from amino acids 151-164 of human NRG1) as follows: ¹⁵¹ESPIRISVSTEGAN¹⁶⁴, preferably the peptide of SEQ ID NO: 2 derived from NRG1 (peptide ranging from amino acids 154-168 of human NRG1) as follows: ¹⁵⁴IRISV¹⁵⁸.

In one particular embodiment, the invention relates to an isolated neutralizing monoclonal antibody that specifically binds to the human neuregulin 1 (NRG1) comprising an heavy chain variable region comprising SEQ ID NO: 3 in the H-CDR1 region, SEQ ID NO: 4 in the H-CDR2 region and SEQ ID NO: 5 in the H-CDR3 region; and a light chain variable region comprising SEQ ID NO: 6 in the L-CDR1 region, SEQ ID NO: 7 in the L-CDR2 region and SEQ ID NO: 8 in the L-CDR3 region.

The inventors have also cloned and characterized the variable domain of the light and heavy chains of said mAb 7E3, and thus determined the complementarity determining regions (CDRs) of said antibody (Table A):

TABLE A
Sequences of mAb 7E3 domains.

| mAb 7E3 | |
|---|---|
| domains | Sequences |
| H-CDR1 | SEQ ID NO: 3 |
| | GYAFTTYL |
| H-CDR2 | SEQ ID NO: 4 |
| | INPEIGKT |
| H-CDR3 | SEQ ID NO: 5 |
| | AREGDYGSSHFAY |
| L-CDR1 | SEQ ID NO: 6 |
| | QSIVYSNGITY |
| L-CDR2 | SEQ ID NO: 7 |
| | KVS |
| L-CDR3 | SEQ ID NO: 8 |
| | FQGSHVPLT |

Accordingly, in a particular embodiment, the invention relates to an isolated monoclonal antibody comprising a heavy chain, wherein the variable domain comprises at least one CDR having a sequence selected from the group consisting of SEQ ID NO: 3 for H-CDR1, SEQ ID NO: 4 for H-CDR2 and SEQ ID NO: 5 for H-CDR3.

In another particular embodiment, the invention relates an isolated monoclonal antibody comprising a light chain, wherein the variable domain comprises at least one CDR having a sequence selected from the group consisting of SEQ ID NO: 6 for L-CDR1, SEQ ID NO: 7 for L-CDR2 and SEQ ID NO: 8 for L-CDR3.

The monoclonal antibody of the invention may comprise a heavy chain wherein the variable domain comprises at least one CDR having a sequence selected from the group consisting of SEQ ID NO: 3 for H-CDR1, SEQ ID NO: 4 for H-CDR2 and SEQ ID NO: 5 for H-CDR3 and a light chain wherein the variable domain comprises at least one CDR having a sequence selected from the group consisting of SEQ ID NO: 6 for L-CDR1, SEQ ID NO: 7 for L-CDR2 and SEQ ID NO: 8 for L-CDR3.

In particular, the invention provides an monoclonal antibody comprising an heavy chain variable region comprising SEQ ID NO: 3 in the H-CDR1 region, SEQ ID NO: 4 in the H-CDR2 region and SEQ ID NO: 5 in the H-CDR3 region; and a light chain variable region comprising SEQ ID NO: 6 in the L-CDR1 region, SEQ ID NO: 7 in the L-CDR2 region and SEQ ID NO: 8 in the L-CDR3 region.

In another embodiment, the monoclonal antibody of the invention is a chimeric antibody, preferably a chimeric mouse/human antibody.

In particular, said mouse/human chimeric antibody may comprise the variable domains of mAb 7E3 antibody as defined above.

In another embodiment, the monoclonal of the invention is a humanized antibody. In particular, in said humanized antibody, the variable domain comprises human acceptor frameworks regions, and optionally human constant domain where present, and non-human donor CDRs, such as mouse CDRs as defined above.

The invention further provides fragments of said antibodies which include but are not limited to Fv, Fab, F(ab′)2, Fab′, dsFv, scFv, sc(Fv)2 and diabodies.

In one embodiment, the anti-NRG1 antibody binds to the ECD of NRG1 with a dissociation constant (Kd) of 1 nM or less. The affinity of the anti-NRG1 antibody is measured by a surface plasmon resonance assay (BIAcore™ assay).

In another aspect, the invention relates to a polypeptide which has a sequence selected from the group consisting of SEQ ID NO: 3, SEQ ID NO: 4, SEQ ID NO: 5; SEQ ID NO: 6; SEQ ID NO: 7 and SEQ ID NO: 8.

In another aspect, the invention provides a monoclonal antibody that competes for binding to human NRG1 with the monoclonal anti-NRG1 antibody 7E3 as defined above.

In a particular embodiment, the invention provides a monoclonal antibody that competes for binding to peptides derived from human NRG1 of SEQ ID NO: 1 or SEQ ID NO: 2 with the monoclonal anti-NRG1 antibody 7E3 as defined above.

Competitive Binding Assays:

The invention thus relates to an isolated monoclonal antibody that specifically binds to human NRG1, competes for binding to human NRG1 with the monoclonal anti-NRG1 antibody 7E3 of the invention as defined above and inhibits NRG1-induced activation of HER3 receptor signaling but without interfering or blocking with NRG1 binding to HER3.

In a particular embodiment, the invention provides a monoclonal antibody that competes for binding to peptides of SEQ ID NO: 1 or SEQ ID NO: 2 the monoclonal anti-NRG1 antibody 7E3 of the invention.

Epitope binning can be used to identify antibodies that fall within the scope of the claimed invention. Epitope binning refers to the use of competitive binding assays to identity pairs of antibodies that are, or are not, capable of binding human NRG1 simultaneously, thereby identifying pairs of antibodies that bind to the same or overlapping epitopes on human NRG1. Epitope binning experiments provide evidence that antigenically distinct epitopes are present. Competition for binding can be evaluated for any pair of antibodies or fragments. For example, using the appropriate detection reagents, the binding specificity of antibodies or binding fragments from any source can be compared to the binding specificity of the monoclonal antibodies disclosed herein. Epitope binning can be performed with “isolated antibodies” or with cell culture supernatants. Frequently, binning is performed with first round clonal supernatants to guide the choice of clones to be developed further. The antibodies to be compared should be substantially homogeneous antigen binding domains. In the case of “bispecific” or “bifunctional” antibodies the binding specificity of the two different binding sites need to be evaluated or binned independently.

The antibodies of the invention may be assayed for specific binding by any method known in the art. Many different competitive binding assay format(s) can be used for epitope binning The immunoassays which can be used include, but are not limited to, competitive assay systems using techniques such western blots, radioimmunoassays, ELISA, “sandwich” immunoassays, immunoprecipitation assays, precipitin assays, gel diffusion precipitin assays, immunoradiometric assays, fluorescent immunoassays, protein A immunoassays, and complement-fixation assays. Such assays are routine and well known in the art (see, e.g., Ausubel et al., eds, 1994 Current Protocols in Molecular Biology, Vol. 1, John Wiley & sons, Inc., New York). For example, the BIACORE® (GE Healthcare, Piscaataway, N.J.) is one of a variety of surface plasmon resonance assay formats that are routinely used to epitope bin panels of monoclonal antibodies. Additionally, routine cross-blocking assays such as those described in Antibodies, A Laboratory Manual, Cold Spring Harbor Laboratory, Ed Harlow and David Lane, 1988, can be performed.

Methods of Producing Antibodies of the Invention:

Anti-NRG1 antibodies of the invention may be produced by any technique known in the art, such as, without limitation, any chemical, biological, genetic or enzymatic technique, either alone or in combination.

Knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies of the invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

So, a further aspect of the invention relates to a vector comprising a nucleic acid of the invention.

Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used, so long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like.

Other examples of plasmids include replicating plasmids comprising an origin of replication, or integrative plasmids, such as for instance pUC, pcDNA, pBR, and the like.

Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. No. 5,882,877, U.S. Pat. No. 6,013,516, U.S. Pat. No. 4,861,719, U.S. Pat. No. 5,278,056 and WO 94/19478.

A further aspect of the invention relates to a host cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids of the invention may be used to produce an antibody of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E.coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (“DHFR gene”) is defective (Urlaub G et al; 1980), rat YB2/3HL.P2.G11.16Ag.20 cell (ATCC CRL1662, hereinafter referred to as “YB2/0 cell”), and the like.

The invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the invention.

Antibodies of the invention are suitably separated from the culture medium by conventional immunoglobulin purification procedures such as protein A-Sepharose, hydroxylapatite chromatography, gel electrophoresis, dialysis, or affinity chromatography.

Amino acid sequence modification(s) of the antibodies described herein are contemplated. For example, it may be desirable to improve the binding affinity and/or other biological properties of the antibody. Modifications and changes may be made in the structure of the antibodies of the invention, and in the DNA sequences encoding them, and still obtain a functional molecule that encodes an antibody with desirable characteristics.

In making the changes in the amino sequences, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

A further aspect of the invention also encompasses function-conservative variants of the antibodies of the invention.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical) over the whole length of the shorter sequence. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the invention, or corresponding DNA sequences which encode said antibodies, without appreciable loss of their biological activity.

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

Accordingly, the invention also provides an antibody comprising a heavy chain wherein the variable domain comprises:

a H-CDR1 having at least 90% or 95% identity with sequence set forth as SEQ ID NO: 3,

a H-CDR2 having at least 90% or 95% identity with sequence set forth as SEQ ID NO: 4,

a H-CDR3 having at least 90% or 95% identity with sequence set forth as SEQ ID NO: 5,

a L-CDR1 having at least 90% or 95% identity with sequence set forth as SEQ ID NO: 6,

a L-CDR2 having at least 90% or 95% identity with sequence set forth as SEQ ID NO: 7,

a L-CDR3 having at least 90% or 95% identity with sequence set forth as SEQ ID NO: 8, and

that specifically binds to human NRG1 with substantially the same affinity as an antibody comprising a heavy chain wherein the variable domain comprises SEQ ID NO: 3 for H-CDR1, SEQ ID NO: 4 for H-CDR2 and SEQ ID NO: 5 for H-CDR3 and a light chain wherein the variable domain comprises SEQ ID NO: 6 for L-CDR1, SEQ ID NO: 7 for L-CDR2 and SEQ ID NO: 8 for L-CDR3.

Said antibodies may be assayed for specific binding by any method known in the art. Many different competitive binding assay format(s) can be used for epitope binning The immunoassays which can be used include, but are not limited to, competitive assay systems using techniques such western blots, radioimmunoassays, ELISA, “sandwich” immunoassays, immunoprecipitation assays, precipitin assays, gel diffusion precipitin assays, immunoradiometric assays, fluorescent immunoassays, protein A immunoassays, and complement-fixation assays. Such assays are routine and well known in the art (see, e.g., Ausubel et al., eds, 1994 Current Protocols in Molecular Biology, Vol. 1, John Wiley & sons, Inc., New York). For example, the BIACORE® (GE Healthcare, Piscaataway, N.J.) is one of a variety of surface plasmon resonance assay formats that are routinely used to epitope bin panels of monoclonal antibodies. Additionally, routine cross-blocking assays such as those described in Antibodies, A Laboratory Manual, Cold Spring Harbor Laboratory, Ed Harlow and David Lane, 1988, can be performed.

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention. Another type of framework modification involves mutating one or more residues within the framework region, or even within one or more CDR regions, to remove T cell—epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 by Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by ldusogie et al.

In another embodiment, one or more amino acid residues are altered to thereby alter the ability of the antibody to fix complement. This approach is described further in PCT Publication WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fc receptor by modifying one or more amino acids. This approach is described further in PCT Publication WO 00/42072 by Presta. Moreover, the binding sites on human IgGI for FcγRI, FcγRII, FcγRIII and FcRn have been mapped and variants with improved binding have been described (see Shields, R. L. et al., 2001 J. Biol. Chen. 276:6591-6604, WO2010106180).

In still another embodiment, the glycosylation of an antibody is modified. For example, an aglycoslated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1 ,176,195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in one embodiment, the antibodies of the invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html).

Alternatively, the antibodies of the invention can be produced in yeasts or filamentous fungi engineered for mammalian-like glycosylation pattern and capable of producing antibodies lacking fucose as glycosylation pattern (see for example EP1297172B1).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP0154316 by Nishimura et al. and EP0401384 by Ishikawa et al.

Another modification of the antibodies that is contemplated by the invention is a conjugate or a protein fusion of at least the antigen-binding region of the antibody of the invention to serum protein, such as human serum albumin or a fragment thereof to increase half-life of the resulting molecule. Such approach is for example described in Ballance et al. EP0322094. Another possibility is a fusion of at least the antigen-binding region of the antibody of the invention to proteins capable of binding to serum proteins, such human serum albumin to increase half-life of the resulting molecule. Such approach is for example described in Nygren et al., EP 0 486 525.

Immunoconjugates:

An antibody of the invention can be conjugated with a detectable label to form an anti-NRG1 immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below. The detectable label can be a radioisotope that is detected by autoradiography. Isotopes that are particularly useful for the purpose of the invention are ³H, ¹²⁵I, ¹³¹I, ³⁵S and ¹⁴C.

Anti-NRG1 immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine.

Alternatively, anti-NRG1 immunoconjugates can be detectably labeled by coupling an antibody to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label anti-NRG1 immunoconjugates of the invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-NRG1 immunoconjugates can be detectably labeled by linking an anti-NRG1 antibody to an enzyme. When the anti-NRG1-enzyme conjugate is incubated in the presence of the appropriate substrate, the enzyme moiety reacts with the substrate to produce a chemical moiety which can be detected, for example, by spectrophotometric, fluorometric or visual means. Examples of enzymes that can be used to detectably label polyspecific immunoconjugates include β-galactosidase, glucose oxidase, peroxidase and alkaline phosphatase.

Those of skill in the art will know of other suitable labels which can be employed in accordance with the invention. The binding of marker moieties to anti-NRG1 monoclonal antibodies can be accomplished using standard techniques known to the art. Typical methodology in this regard is described by Kennedy et al., Clin. Chim. Acta 70:1, 1976; Schurs et al., Clin. Chim. Acta 81:1, 1977; Shih et al., Int'l J. Cancer 46:1101, 1990; Stein et al., Cancer Res. 50:1330, 1990; and Coligan, supra.

Moreover, the convenience and versatility of immunochemical detection can be enhanced by using anti-NRG1 monoclonal antibodies that have been conjugated with avidin, streptavidin, and biotin. (See, e.g., Wilchek et al. (eds.), “Avidin-Biotin Technology,” Methods In Enzymology (Vol. 184) (Academic Press 1990); Bayer et al., “Immunochemical Applications of Avidin-Biotin Technology,” in Methods In Molecular Biology (Vol. 10) 149-162 (Manson, ed., The Humana Press, Inc. 1992).)

Methods for performing immunoassays are well-established. (See, e.g., Cook and Self, “Monoclonal Antibodies in Diagnostic Immunoassays,” in Monoclonal Antibodies: Production, Engineering, and Clinical Application 180-208 (Ritter and Ladyman, eds., Cambridge University Press 1995); Perry, “The Role of Monoclonal Antibodies in the Advancement of Immunoassay Technology,” in Monoclonal Antibodies: Principles and Applications 107-120 (Birch and Lennox, eds., Wiley-Liss, Inc. 1995); Diamandis, Immunoassay (Academic Press, Inc. 1996).)

In another aspect, the invention provides an anti-NRG1 antibody-drug conjugate. An “anti-NRG1 antibody-drug conjugate” as used herein refers to an anti-NRG1 antibody according to the invention conjugated to a therapeutic agent. Such anti-NRG1 antibody-drug conjugates produce clinically beneficial effects on NRG1-expressing cells when administered to a patient, such as, for example, a patient with a NRG1-expressing cancer, typically when administered alone but also in combination with other therapeutic agents.

In typical embodiments, an anti-NRG1 antibody is conjugated to a cytotoxic agent, such that the resulting antibody-drug conjugate exerts a cytotoxic or cytostatic effect on a NRG1-expressing cell (e.g., a NRG1-expressing cancer cell) when taken up or internalized by the cell. Particularly suitable moieties for conjugation to antibodies are chemotherapeutic agents, prodrug converting enzymes, radioactive isotopes or compounds, or toxins. For example, an anti-NRG1 antibody can be conjugated to a cytotoxic agent such as a chemotherapeutic agent or a toxin (e.g., a cytostatic or cytocidal agent such as, for example, abrin, ricin A, pseudomonas exotoxin, or diphtheria toxin).

Useful classes of cytotoxic agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and- carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like.

Individual cytotoxic agents include, for example, an androgen, anthramycin (AMC), asparaginase, 5-azacytidine, azathioprine, bleomycin, busulfan, buthionine sulfoximine, camptothecin, carboplatin, carmustine (BSNU), CC-1065 (Li et al., Cancer Res. 42:999-1004, 1982), chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, etopside phosphate (VP-16), 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide (VM-26), 6-thioguanine, thioTEPA, topotecan, vinblastine, vincristine, and vinorelbine.

Particularly suitable cytotoxic agents include, for example, dolastatins (e.g., auristatin E, AFP, MMAF, MMAE), DNA minor groove binders (e.g., enediynes and lexitropsins), duocarmycins, taxanes (e.g., paclitaxel and docetaxel), puromycins, vinca alkaloids, CC-1065, SN-38 (7-ethyl-10-hydroxy-camptothein), topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone. In certain embodiments, a cytotoxic agent is a conventional chemotherapeutic such as, for example, doxorubicin, paclitaxel, melphalan, vinca alkaloids, methotrexate, mitomycin C or etoposide. In addition, potent agents such as CC-1065 analogues, calicheamicin, maytansine, analogues of dolastatin 10, rhizoxin, and palytoxin can be linked to an anti-NRG1 antibody.

In specific variations, the cytotoxic or cytostatic agent is auristatin E (also known in the art as dolastatin-10) or a derivative thereof. Typically, the auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatin derivatives include AFP (dimethylvaline-valine-dolaisoleuine-dolaproine-phenylalanine-p-phenylenediamine), MMAF (dovaline-valine-dolaisoleunine-dolaproine-phenylalanine), and MAE (monomethyl auristatin E). The synthesis and structure of auristatin E and its derivatives are described in U.S. Patent Application Publication No. 20030083263; International Patent Publication Nos. WO 2002/088172 and WO 2004/010957; and U.S. Pat. Nos. 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In other variations, the cytotoxic agent is a DNA minor groove binding agent. (See, e.g., U.S. Pat. No. 6,130,237.) For example, in certain embodiments, the minor groove binding agent is a CBI compound. In other embodiments, the minor groove binding agent is an enediyne (e.g., calicheamicin).

In certain embodiments, an antibody-drug conjugate comprises an anti-tubulin agent. Examples of anti-tubulin agents include, for example, taxanes (e.g., Taxol® (paclitaxel), Taxotere® (docetaxel)), T67 (Tularik), vinca alkyloids (e.g., vincristine, vinblastine, vindesine, and vinorelbine), and dolastatins (e.g., auristatin E, AFP, MMAF, MMAE, AEB, AEVB). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin. In some embodiments, the cytotoxic agent is a maytansinoid, another group of anti-tubulin agents. For example, in specific embodiments, the maytansinoid is maytansine or DM-1 (ImmunoGen, Inc.; see also Chari et al., Cancer Res. 52:127-131, 1992).

In other embodiments, the cytotoxic agent is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, or trifluridine.

In other embodiments, an anti-NRG1 antibody is conjugated to a pro-drug converting enzyme. The pro-drug converting enzyme can be recombinantly fused to the antibody or chemically conjugated thereto using known methods. Exemplary pro-drug converting enzymes are carboxypeptidase G2, β-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.)

Diagnostic Uses:

A further aspect of the invention relates to an anti-NRG1 antibody of the invention for diagnosing and/or monitoring a cancer disease and other diseases in which NRG1 levels are modified (increased or decreased).

In a preferred embodiment, antibodies of the invention may be labelled with a detectable molecule or substance, such as a fluorescent molecule, a radioactive molecule or any others labels known in the art as above described. For example, an antibody of the invention may be labelled with a radioactive molecule by any method known to the art. For example radioactive molecules include but are not limited radioactive atom for scintigraphic studies such as I¹²³, I¹²⁴, In1¹¹, Re¹⁸⁶, Re¹⁸⁸. Antibodies of the invention may be also labelled with a spin label for nuclear magnetic resonance (NMR) imaging (also known as magnetic resonance imaging, MRI), such as iodine-123, iodine-131, indium-111, fluorine-19, carbon-13, nitrogen-15, oxygen-17, gadolinium, manganese or iron. Following administration of the antibody, the distribution of the antibody within the patient is detected. Methods for detecting distribution of any specific label are known to those skilled in the art and any appropriate method can be used. Some non-limiting examples include, computed tomography (CT), position emission tomography (PET), magnetic resonance imaging (MRI), fluorescence, chemiluminescence and sonography.

Antibodies of the invention may be useful for diagnosing and staging of cancer diseases associated with NRG1 overexpression. Cancer diseases associated with NRG1 overexpression typically include but are not limited lung cancer, especially non-small cell lung cancer (NSCLC), pancreatic cancer and bladder cancer.

Typically, said diagnostic methods involve use of biological sample obtained from the patient. As used herein the term “biological sample” encompasses a variety of sample types obtained from a subject and can be used in a diagnostic or monitoring assay. Biological samples include but are not limited to blood and other liquid samples of biological origin, solid tissue samples such as a biopsy specimen or tissue cultures or cells derived therefrom, and the progeny thereof. For example, biological samples include cells obtained from a tissue sample collected from an individual suspected of having a cancer disease associated with NRG1 overexpression, and in a preferred embodiment from lung cancer, especially non-small cell lung cancer (NSCLC), pancreatic cancer and bladder cancer.

Therefore, biological samples encompass clinical samples, cells in culture, cell supernatants, cell lysates, serum, plasma, biological fluid, and tissue samples.

Therapeutic Uses:

Antibodies, fragments or immunoconjugates of the invention may be useful for treating any diseases associated with NRG1 overexpression preferentially cancers. The antibodies of the invention may be used alone or in combination with any suitable agent.

An anti-NRG1 antibody of the invention may be used as treatment of hyperproliferative diseases associated with NRG1 overexpression.

Examples of such diseases associated with NRG1 overexpression encompasses lung cancer, especially non-small cell lung cancer (NSCLC), pancreatic cancer and bladder cancer.

In each of the embodiments of the treatment methods described herein, the anti-NRG1 antibody or anti-NRG1 antibody-drug conjugate is delivered in a manner consistent with conventional methodologies associated with management of the disease or disorder for which treatment is sought. In accordance with the disclosure herein, an effective amount of the antibody or antibody-drug conjugate is administered to a patient in need of such treatment for a time and under conditions sufficient to prevent or treat the disease or disorder.

Thus, an aspect of the invention relates to a method for treating a disease associated with the overexpression of NRG1 comprising administering a patient in need thereof with a therapeutically effective amount of an antibody, fragment or immunoconjugate of the invention.

In this context, the term “treating” or “treatment”, as used herein, means reversing, alleviating, inhibiting the progress of, or preventing the disorder or condition to which such term applies, or one or more symptoms of such disorder or condition. According to the invention, the term “patient” or “patient in need thereof” is intended for a human affected or likely to be affected with disease associated with the overexpression of NRG1.

By a “therapeutically effective amount” of the antibody of the invention is meant a sufficient amount of the antibody to treat said disease associated with the overexpression of NRG1 such as a cancer (e.g. pancreatic cancer), at a reasonable benefit/risk ratio applicable to any medical treatment. It will be understood, however, that the total daily usage of the antibodies and compositions of the present invention will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific antibody employed; and like factors well known in the medical arts. For example, it is well known within the skill of the art to start doses of the compound at levels lower than those required to achieve the desired therapeutic effect and to gradually increase the dosage until the desired effect is achieved.

In certain embodiments, an anti-NRG1 antibody or antibody-drug conjugate is used in combination with a second agent for treatment of a disease or disorder. When used for treating cancer, the anti-NRG1 antibody or antibody-drug conjugate of the invention may be used in combination with conventional cancer therapies such as, e.g., surgery, radiotherapy, chemotherapy, or combinations thereof.

Pharmaceutical Compositions:

For administration, the anti-NRG1 antibody or antibody-drug conjugate is formulated as a pharmaceutical composition. A pharmaceutical composition comprising an anti-NRG1 antibody or antibody-drug conjugate can be formulated according to known methods to prepare pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. (See, e.g., Gennaro (ed.), Remington's Pharmaceutical Sciences (Mack Publishing Company, 19th ed. 1995)) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, parenteral, intranasal, intravenous, intramuscular, subcutaneous or intraocular administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the antibody may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An antibody of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibodies of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

Kits:

Finally, the invention also provides kits comprising at least one antibody of the invention. Kits containing antibodies of the invention find use in detecting NRG1 expression (increase or decrease), or in therapeutic or diagnostic assays. Kits of the invention can contain an antibody coupled to a solid support, e.g., a tissue culture plate or beads (e.g., sepharose beads). Kits can be provided which contain antibodies for detection and quantification of NRG1 in vitro, e.g. in an ELISA or a Western blot. Such antibody useful for detection may be provided with a label such as a fluorescent or radiolabel.

The invention will be further illustrated by the following figures and examples. However, these examples and figures should not be interpreted in any way as limiting the scope of the present invention.

FIGURES:

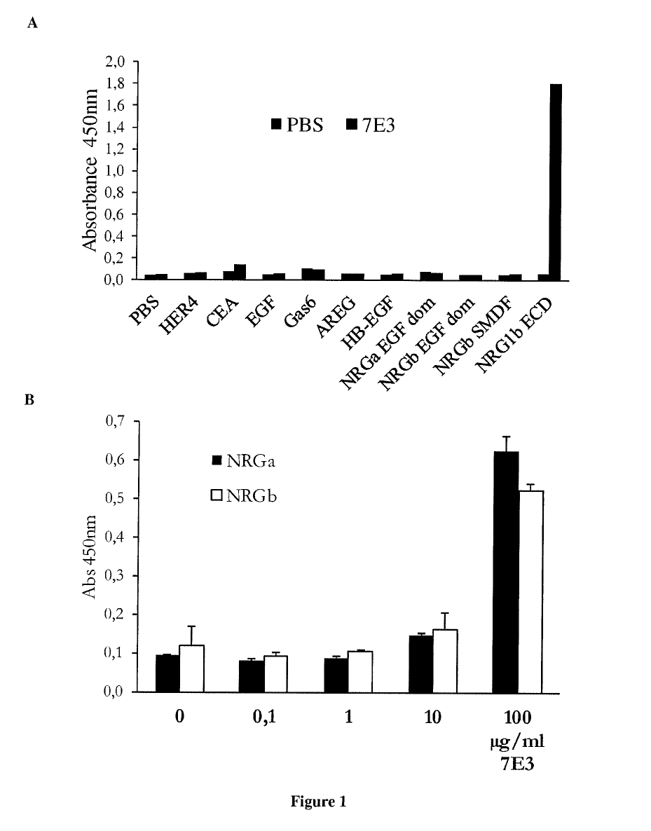

FIG. 1: Elisa assay of specificity of anti-NRG mAb (7E3). Plates coated with human HER4, CEA, EGF, Gas-6, AREG, HB-EGF, NRG alpha (EGF-domain), NRG beta (EGF domain), NRG SMDF and NRG1 beta (ExtraCellular Domain) (A) or ExtraCellular Domain of NRG alpha and beta (B) were incubated with purified murine anti-NRG mAb or 35A7 (anti-CEA antibody) or a commercial Ab. After washing, HRP-conjugated anti-mouse IgG was added. 7E3 and 18A10 anti-NRG mAb binds specifically to NRG1b ECD and doesn't cross-react with other ligands and EGF-domain of NRG beta and alpha.

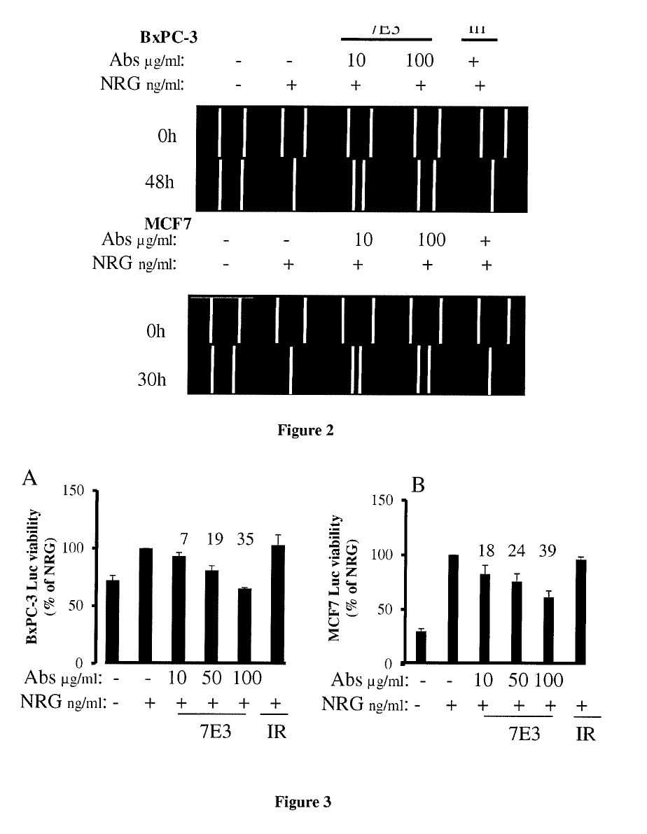

FIG. 2: Wound healing assay to investigate the effect of 7E3 anti-NRG Ab on cellular migration. After grown to confluency, BxPC-3 and MCF7 HER3 positive cells were starved and wounded with a pipette tip. Cells were incubated with 25 (BxPC-3) or 12 ng/ml (MCF7) of NRG1 and increasing doses of 7E3. 7E3 Ab reduced the repopulation of the cleared area at 10 and 100 microg/ml more significantly than the irrelevant Ab, even though the cells were treated with NRG.

FIG. 3: Cell viability assay to investigate the anti-proliferative efficacy of anti-NRG 7E3 Ab. BxPC-3 and MCF7 cells transfected with the luciferase gene were grown in serum free medium and treated with 15 ng/ml or 10 ng/ml of NRG1 for BXPC-3-luc and MCF7-luc respectively. 10, 50 and 100 microg/ml of 7E3 Ab were added for 5 days. The irrelevant Ab was added at 100 microg/ml. Bioluminescence was measured and the % of viability calculated. An inhibition of cell viability was observed in cells treated with 7E3 Ab in comparison with cells treated with NRG1 or irrelevant Ab.

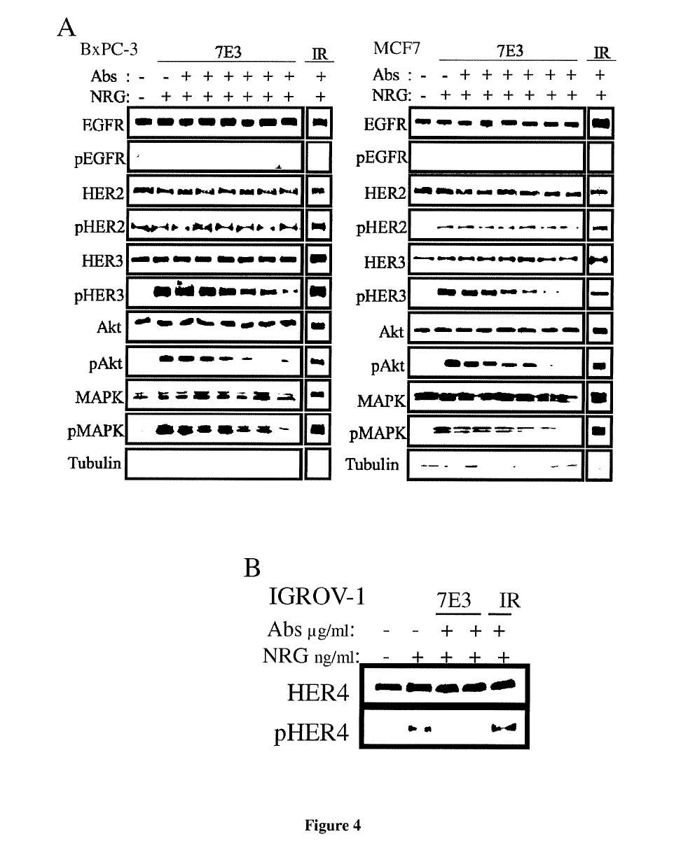

FIG. 4: Effect of 7E3 antibody on HER signaling pathways by western blot. (A) BxPC-3, MCF7 and IGROV-1 were incubated with NRG1 (15 ng/ml for BxPC-3, 10 ng/ml for MCF7, 25 ng/ml for IGROV-1) and 7E3 or irrelevant Ab (10 or 100 microg/ml) for IGROV-1 and (6 to 100 microg/ml) for 15 minutes for BxPC-3 and MCF7. 7E3 Ab induced an inhibition of pHER3, pAKT and pMAK in contrast no effect has been observed on pEGFR and HER2. (B) 7E3 is able to inhibit the phosphorylation of HER4 in IGROV-1 cell line.

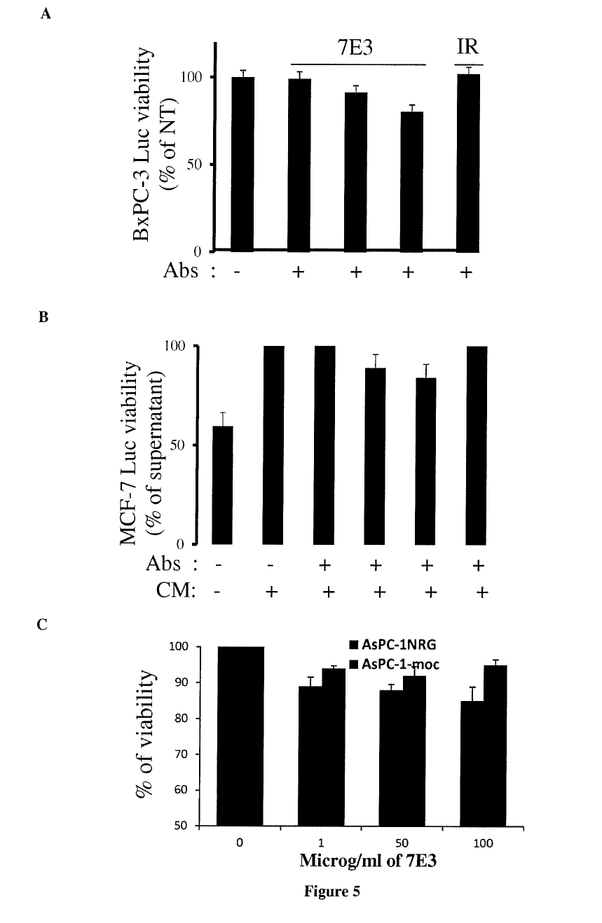

FIG. 5: Proliferation assay of (A) 7E3 Ab on NRG1 secreting BxPC-3 cells or (B) on NRG1 non-secreting MCF7 incubated with conditioned medium (CM) of BxPC-3 or (C) on AsPC-1 WT or AsPC-1 transfected with NRG1. Cells were grown in 1% SVF and incubated with antibodies (10, 50 and 100 microg/ml) for 5 days. 7E3 Ab inhibited the viability of NRG1 expressing BxPC-3 and AsPC1-NRG. An inhibition of viability of MCF7 cells (not expressing NRG1) incubated with CM of BxPC-3 is observed.

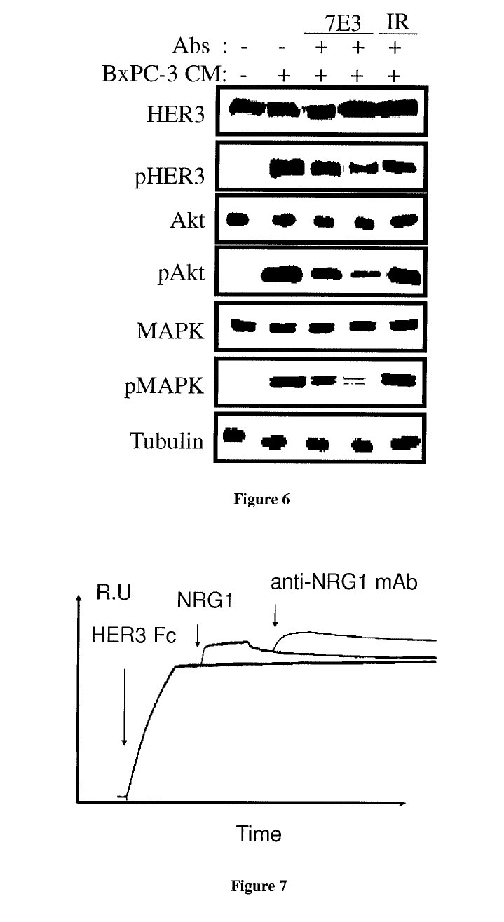

FIG. 6: Effect of 7E3 Ab on NRG non-secreting MCF7 cell lines incubated with Condition Medium of BxPC-3. MCF7 were incubated 15 minutes with supernatant of NRG1 secreting BxPC-3 grown in starved medium for 48 h. 7E3 Ab was added at 10 or 100 microg/ml for 15 minutes. Irrelevant Ab was added at 100 microg/ml. An inhibition of phosphorylation of HER3, AKT and MAPK is observed.

FIG. 7: 7E3 is able to bind the NRG already linked to HER3. Biacore analysis: chip was coated with anti-Fc antibody, then HER3-Fc molecule (25 microg/ml) is injected in the flow and bind anti-Fc antibody. Then NRG (185 nM) is injected and finally 7E3 antibody (200 nM).

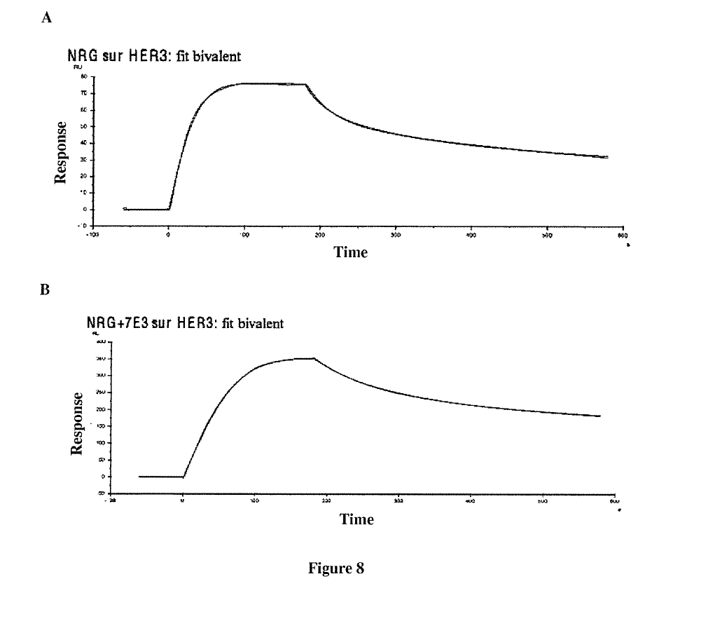

FIG. 8: 7E3/NRG complex is able to bind HER3 with a similar affinity than NRG1 alone on HER3. Biacore analysis: anti-Fc Ab was coated on chip, then HER3-fc receptor is injected in the flow and bind to antibody. (A) NRG is injected and bind HER3 or (B) a mixture of NRG (150 mM) plus Ab (200 nM) is injected and is able to bind HER3.

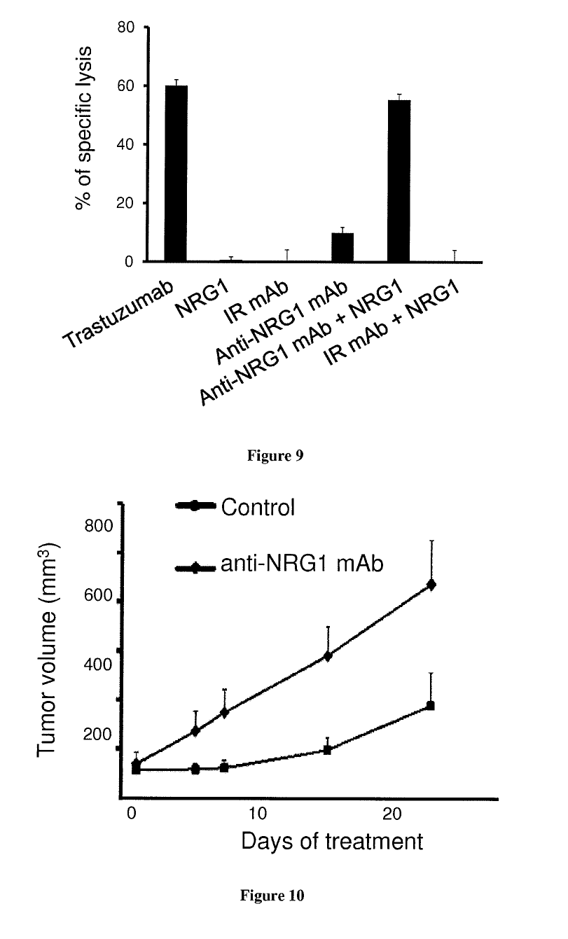

FIG. 9: ADCC analysis. Antibody Dependant Cell Cytotoxicity (ADCC, Kit Promega LDH release) was studied on BxPC-3 incubated with human PBMC (ratio 1/15) and NRG1 mAb or irrelevant mAb (10 mg/ml) in presence or not of NRG1. Any ADCC was observed on BxPC-3 shHER3.

FIG. 10: In vivo experiments. BxPC-3 cells were xenografted in athymic nude mice and when tumor volume reached 150 mm 3 mice were treated either with anti-NRG1 mAb or with irrelevant mAb (10 mg/kg, 2/week, for 1 month). A significant growth inhibition was observed in the group treated with anti-NRG1 mAb (p=0.064).

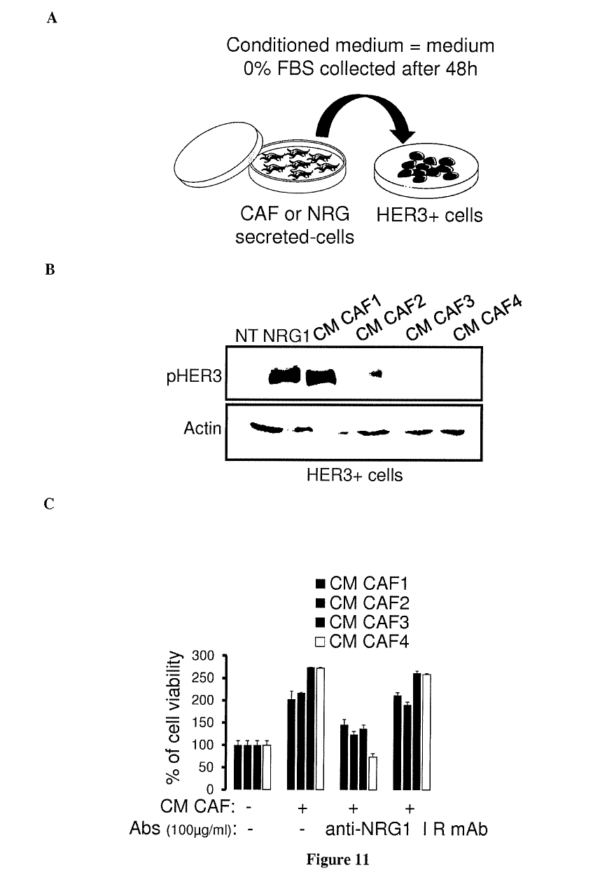

FIG. 11: Cancer Associated Fibroblasts and NRG1. (A) Conditioned medium (CM) is obtained after incubation of CAF 48 h with serum free medium. CAF came from pancreatic tumors removed in patients. (B) Four CAF's CM samples are able to phosphorylate HER3 receptor after 15 min of incubation, this result strongly indicates the secretion of NRG1 by CAF. (C) This conditioned medium is incubated on BxPC-3 and mAbs for 5 days. Cell proliferation is analyzed by SRB assay.

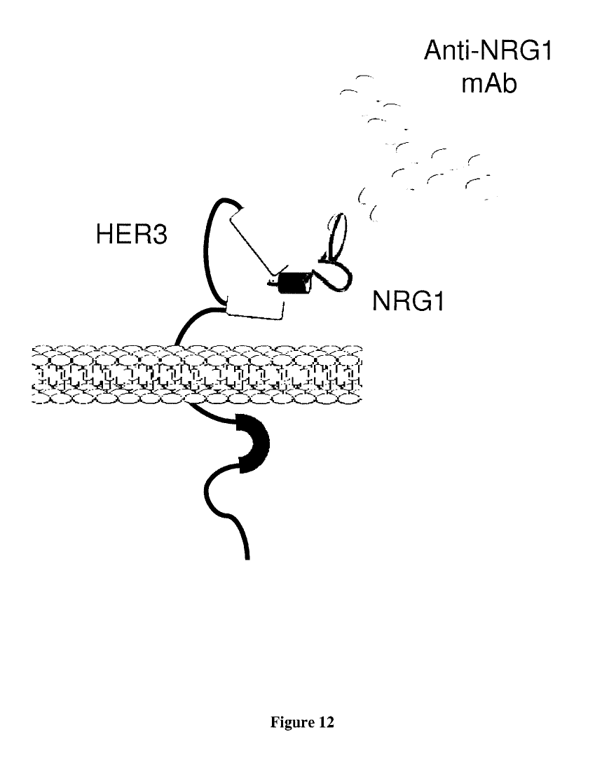

FIG. 12: Characterization of the 7E3 anti-NRG1 antibody. NRG1 binds HER3 receptor thanks to EGF domain, 7E3 mAb's epitope is localized on NRG's IgG like domain as determined by ELISPOT and Alascan analysis.

### EXAMPLE

Material & Methods

Cell lines and reagents: Neuregulin 1 beta 1 extracellular domain (ECD) (NRG1β1) was purchased from RD Systems (Minneapolis, Minn.). The BxPC-3 (pancreas) and MCF7 (breast) cell lines were obtained from ATCC (Rockville, Md., USA). Cells were cultured in RPMI 1640 supplemented as recommended by ATCC, usually with 10% FCS. Cells were grown at 37° C. in a humidified atmosphere of 5% CO2 and medium was replaced twice a week. Cells are used within 3 months from a master cell bank. Routine authentication by typical morphology observation and myco-plasma test were conducted using MycoAlert mycoplasma detection kit (Lonza, Basel, Switzerland). Luciferase-positive BxPC-3 and MCF7 (BxPC-3-Luc, MCF7-Luc) were generated in the laboratory.

ELISA assay: 96-well microtiter plates were coated with human HER4, CEA, EGF, Gas-6, AREG, HB-EGF, NRG alpha (EGF-domain), NRG beta (EGF domain), NRG SMDF and NRG1 beta (ExtraCellular Domaine) or Extra cellular domain of NRG alpha and beta in PBS ON at 4° C. After washing and incubation with PBS-BSA (1 mg/ml), purified murine anti-NRG mAb or PBS were added in each well 1 hour. After washing, HRP-conjugated anti-mouse IgG was added 1 hour followed by OPD and the OD is measured at 450 nm.

Cell migration (wound healing) assay: 500,000 cells were seeded in 6-well plates and grown at 37° C. in RPMI medium with 10% FBS. After 24 h, cells were incubated with serum free medium for 24 h and at 90% confluence, a wound was generated by scratching each monolayer with a pipette tip. Cells were then incubated in medium with 25 ng/ml of NRG1b and/or with 100 μg/ml anti-NRG1 mAb. Cell migration was observed 24 h after and then captured by a Nikon ECLIPSE TS100 microscope and an Olympus SP-510 UZ camera.

Cell proliferation assay: The effect of NRG1β1 and 7E3 on cell proliferation was evaluated using luciferase-activity assay. 9000 cells per well were seeded in 96-well microtiter plates. After 24 h, cells were incubated with serum free medium for 24 h. 10 (MCF7) or 15 ng/ml (BxPC-3) of NRG1b and 10-100 μg/ml of anti-NRG1 mAb were added. After 5 days of incubation at 37° C., the supernatant was removed and luciferine (Promega, Wis., USA) substrate added on the cells. Bioluminescence was determined using the Wallac Trilux 1450 Microbeta liquid scintillation and luminescence counter (Perkin-Elmer, MA, USA). Growth inhibition was calculated based on the percentage of proliferating cells in treated samples relative to untreated cultures. All experiments were performed five times.

Western blot analysis: BxPC3 and MCF7 tumor cells were plated at 2 000,000 cells/well in culture plates and cultured at 37° C. for 24 hours. After serum starvation in RPMI serum free medium for 16 hours, cells were washed and incubated in 15 ng/ml (BxPC-3) or 10 ng/ml (MCF7) of NRG1b and/or anti-NRG1b mAb for 15 min. Cells were then washed, scraped, and lysed with buffer containing 20 mM Tris-HCl (pH 7.5), 150 mM NaCl, 1.5 mM MgCl2,1 mM EDTA, 1% Triton, 10% glycerol, 0.1 mM phenylmethylsulfonyl fluoride, 100 mM sodium fluoride, 1 mM sodium orthovanadate (Sigma-Aldrich), and one tablet of complete protease inhibitor mixture (Roche Diagnostics, Indianapolis, Ind.). After 30 minutes, the insoluble fraction was eliminated by centrifugation, protein concentrations determined and western blotting performed.

Membranes were incubated with the anti-human HER3 (Millipore, Billerica, Mass.) and anti-human EGFR, HER2, ERK1/2, AKT, or anti-phosphorylated EGFR, HER3, HER2, ERK1/2 or AKT antibodies (Cell Signaling Technology, Beverly, Mass.). Equal loading was assessed with an antibody against β-actin (Cell Signaling Technology).

BIAcore analysis: The kinetic parameters of the binding of selected Abs to NRG1 were determined at 20° C. by surface plasmon resonance analysis using a BIAcore 3000 instrument (BIAcore AB, Uppsala, Sweden). 25 μg/ml of HER3-specific Abs were immobilized on the 07-15CM5S sensor chip surface using anti-human Fc Ab (Sigma-Aldrich) according to the manufacturer's instructions. Human recombinant NRG1b in HBS-EP buffer [10 mM Hepes (pH 7.4), 3 mM EDTA, 150 mM NaCl, and 0.005% non-ionic surfactant P20 (GE Healthcare)] was injected at concentrations 150 nM over the flow cell, and 7E3 mAb was injected at 200 nM. The dissociation phase was followed by a regeneration step with MgCl2 3M solution. All sensorgrams were corrected by subtracting the control flow cell signal. Data were globally fitted to a Langmuir 1:1 model using the BIAevaluation version 4.1.1 software.

Analysis of 7E3 mAb binding on NRG/HER3 complex by Immunofluorescence: HER3 positive BxPC-3 or sh-HER3 BxPC-3 cells were plates 24 h in 10% SVF medium. Then cells were starved during 48 h and incubated with increasing doses of NRG1 (0, 25 50 100 ng/ml) and 20 microg/ml of 7E3. An anti-CEA antibody was used as positive control and anti-HER3 antibody was used to show the expression of HER3. Nuclei were labeled with DAPI.

ADCC: Antibody-dependent cellular cytotoxicity (ADCC) assay was evaluated with a luciferase-activity assay. In 96-well white plates, BxPC-3-Luc (10000 cells per well) were pre-incubated with antibodies (7E3 or Px as control) and NRG1b (250 ng/ml) for 30 min. Ficoll-purified human peripheral blood mononuclear cells from buffy coat were then added at a 15:1 effector to target cell ratio (E:T). After 24 h of incubation at 37C, the supernatant was removed and luciferine (Promega, Wis., USA) added on the cells. Bioluminescence was determined using the Wallac Trilux 1450 Microbeta liquid scintillation and luminescence counter (Perkin-Elmer, MA, USA). Percentage of cellular cytotoxicity was calculated using the following formula: percentage of specific lysis ¼ [bioluminescence in experimental point—basal bioluminescence]/[bioluminescence in total lysis—basal bioluminescence]*100. Basal bioluminescence is obtained when BxPC-3-Luc cells are incubated with hPBMC alone and bioluminescence in total lysis is obtained after a 30 min incubation of BxPC-3-Luc with SDS (0.1%).

Tumor xenografts and treatment: All in vivo experiments were performed in compliance with the French regulations and ethical guidelines for experimental animal studies in an accredited establishment (Agreement No. C34-172-27). Six week/old female athymic nude mice, purchased from Harlan (Le Malcourlet, France), were injected subcutaneously into the right flank with BxPC-3 (3.5×106). Tumor-bearing mice were randomized to different treatment groups (at least 8 animals/group) when tumors reached a volume of 100 mm3 and were then treated with anti-NRG1 7E3 or Px mAbs as control [10 mg/kg of each mAb]. Antibodies were given intraperitonally (i.p.) twice a week for 4 weeks. Tumor volumes were measured using a caliper and volume was calculated by using the formula: D1×D2×D3/2. For survival comparison, mice were sacrificed when tumors reached a volume of 1800 mm3. For orthotopic model, BxPC-3-luciferase (1×106) were injected in the pancreas of mice and treatment started one week later with anti-NRG1 7E3 or Px mAbs as control [10 mg/kg of each mAb]. Antibodies were given intraperitonally (i.p.) twice a week for 4 weeks and bioluminescence measured every weeks. At day 48, mice were sacrified and bioluminescence measured in pancreas.

Epitope Analysis:

For SPOT alanine scanning analysis, 39 pentadecapeptides corresponding to the Ab-immunoreactive amino acid sequences previously identified and the 12 alanine analogs of each peptide were synthesized by the SPOT method. Ab reactivity of cellulose-bound peptides was assayed as described above. Spot reactivity was evaluated by scanning the membranes and measuring the spot intensities with ImageJ.

Results

Specificity of anti-NRG mAb (7E3): the anti-NRG1 mAb (7E3) bound specifically to NRG1 beta and alpha as shown by ELISA assay. 7E3 didn't bind the others ligands or proteins such as HER receptors or CEA (FIG. 1). Epitope of anti-NRG mAb (7E3): The epitope of anti-NRG1 antibody (7E3) is IRISV as determined by SPOT alanine scanning analysis. This epitope is in IgG like domain of NRG1.

Functionality of anti-NRG1 mAb (7E3) on cell proliferation and migration: the anti-NRG1 mAb (7E3) inhibited BxPC-3 and MCF7 (HER3 positive) cell migration at 10 or 100 μg/ml following recombinant NRG1 stimulation as compared with irrelevant antibody (FIG. 2). An inhibition of BxPC-3 and MCF7 was observed by cell viability assay using bioluminescence measurement in cells treated by 7E3 with recombinant NRG1. More than 30% of inhibition was observed with 100 μg/ml of antibody (FIG. 3).

Inhibition of HER signaling pathways by anti-NRG1 mAb (7E3): 7E3 induced an inhibition of phosphorylation of HER3 (BxPC-3 and MCF7) and HER4 (IGROV-1) as well as phosphorylation of AKT and MAPK after 15 minutes of mAb treatment and NRG1 (FIG. 4). No effect was observed on pEGFR and pHER2.

The anti-NRG1 mAb (7E3) inhibited proliferation NRG1 secreting BxPC-3 cells (FIG. 5A) as well as NRG1 non-secreting MCF7 incubated with conditioned medium of BxPC-3 (FIG. 5B). 7E3 is functional on Neuregulin 1 secreted by cells. Furthermore an inhibition of AsPC-1 transfected with NRG1 proliferation is observed with 7E3 in comparison with control AsPC-1 (FIG. 5C).

An inhibition of phosphorylation of HER3, AKT and MAPK is shown on NRG non-secreting MCF7 cells incubated with condition medium of BxPC-3 (FIG. 6).

Biacore analysis shown that 7E3 antibody is able to bind NRG1 already linked to HER3 (FIG. 7). Furthermore the 7E3/NRG1 complex has the same affinity for HER3 than NRG1 alone (FIG. 8). Finally, immunofluorescence assay shown the binding of 7E3 on HER3 positive BxPC-3 cells incubated with increased concentrations of recombinant NRG1. In contrast, no signal was observed on shHER3 BxPC-3 demonstrating the specificity of the binding of 7E3 on NRG1/HER3 complex.

ADCC analysis and in vivo efficiency: a specific lysis of BxPC-3 incubated with 7E3, NRG1 and hPBMC is observed by LDH release assay demonstrating the ability of 7E3 to induce an ADCC response on HER3 positive cell lines in presence of NRG1 (FIG. 9).

In BxPC-3 xenografted mice, 7E3 induced a significant tumor growth inhibition as compared with mice treated with irrelevant antibody (FIG. 9).

In an orthotopic model, BxPC-3 cells were grafted in pancreas of nude mice. A significant decrease of bioluminescence in pancreas was observed in mice treated with 7E3 twice a week in comparison with mice treated with irrelevant antibody (p=0.0404).

Cancer associated fibroblasts (CAF) and NRG1: Conditioned medium is obtained after 48 h incubation of CAF with serum free medium. CAF's CM samples are able to phosphorylate HER3 by western blot analysis (FIG. 10) demonstrating the secretion of NRG by CAF.

7E3 mAb incubated with CAF's CM inhibited BxPC-3 proliferation in comparison to CM alone or with irrelevant antibody (FIG. 11).

Expression of EGFR ligands (EGF, HB-EGF, TGFa, NRG1) in several pancreatic cancer cells has been analysed by RT-PCR.

The effect on viability of combination of anti-NRG1 with anti-HB-EGF or anti-TGFa antibody (collaboration with Y. Yarden) has been studied in BxPC-3 model by bioluminescence analysis. The best combination is the association of anti-NRG1 antibody (7E3) with anti-HB-EGF.

### REFERENCES

Throughout this application, various references describe the state of the art to which this invention pertains. The disclosures of these references are hereby incorporated by reference into the present disclosure.

Hegde G V, de la Cruz C C, Chiu C, Alag N, Schaefer G, Crocker L, Ross S, Goldenberg D, Merchant M, Tien J, Shao L, Roth L, Tsai S P, Stawicki S, Jin Z, Wyatt S K, Carano R A, Zheng Y, Sweet-Cordero E A, Wu Y, Jackson E L; Blocking NRG1 and other ligand-mediated Her4 signaling enhances the magnitude and duration of the chemotherapeutic response of non-small cell lung cancer; Sci Transl Med. 2013 Feb. 6; 5(171):171ra18.

Liles J S, Arnoletti J P, Kossenkov A V, Mikhaylina A, Frost A R, Kulesza P, Heslin M J, Frolov A; Targeting ErbB3-mediated stromal-epithelial interactions in pancreatic ductal adenocarcinoma.; Br J Cancer. 2011 Aug. 9; 105(4):523-33.

1. An isolated neutralizing monoclonal antibody that specifically binds to the human neuregulin 1 (NRG1) without interfering with NRG1 binding to HER3 receptor.

2. The antibody according to claim 1, comprising a heavy chain variable region comprising SEQ ID NO: 3 in the H-CDR1 region, SEQ ID NO: 4 in the H-CDR2 region and SEQ ID NO: 5 in the H-CDR3 region; and a light chain variable region comprising SEQ ID NO: 6 in the L-CDR1 region, SEQ ID NO: 7 in the L-CDR2 region and SEQ ID NO: 8 in the L-CDR3 region.

3. The antibody according to claim 1, wherein the antibody specifically binds to the extracellular domain (ECD) of NRG1.

4. A fragment of an antibody according to claim 1, wherein the fragment is selected from the group consisting of Fv, Fab, F(ab′)2, Fab′, dsFv, scFv, sc(Fv)2 and diabodies.

5. A nucleic acid sequence encoding a heavy chain or light chain of an antibody according to claim 1.

6. A vector comprising a nucleic acid according to claim 5.

7. A host cell comprising i) a nucleic acid according to claim 5 or ii) a vector comprising the nucleic acid.

8. A pharmaceutical composition comprising an antibody according to claim 1, or a fragment thereof.

9. The antibody according to claim 1, wherein said antibody is an anti-NRG1 immunoconjugate.

10-12. (canceled)

13. A method for treating a disease associated with the overexpression of NRG1 in a patient in need thereof, comprising
administering to the patient a therapeutically effective amount of an antibody of claim 1 or a fragment thereof, or an immunoconjugate comprising the antibody.

14. The method of claim 13, wherein the disease is cancer.

15. The method of claim 14, wherein the cancer is pancreatic cancer.


SEQUENCE LISTING (8 sequences)

SEQ ID NO: 1 - protein, 14 aa, Artificial Sequence
  Synthetic peptide ranging from amino acids 151-164 of human NRG1
ESPIRISVSTEGAN

SEQ ID NO: 2 - protein, 5 aa, Artificial
  Synthetic peptide ranging from amino acids 154-168 of human NRG1
IRISV

SEQ ID NO: 3 - protein, 8 aa, Artificial
  Synthetic 7E3 H-CDR1
GYAFTTYL

SEQ ID NO: 4 - protein, 8 aa, Artificial
  Synthetic 7E3 H-CDR2
INPEIGKT

SEQ ID NO: 5 - protein, 13 aa, Artificial
  Synthetic 7E3 H-CDR3
AREGDYGSSHFAY

SEQ ID NO: 6 - protein, 11 aa, Artificial
  Synthetic 7E3 L-CDR1
QSIVYSNGITY

SEQ ID NO: 7 - protein, 3 aa, Artificial
  Synthetic 7E3 L-CDR2
KVS

SEQ ID NO: 8 - protein, 9 aa, Artificial
  Synthetic 7E3 L-CDR3
FQGSHVPLT